(19) United States
(12) Patent Application Publication
(10) Pub. No.: US 2009/0069701 A1
(43) Pub. Date: Mar. 12, 2009

(54) Externally-connected expandable blood pressure meter

(75) Inventor: Daniel C.M. Tseng, Taipei County (TW)

Correspondence Address: Joe McKinney Muncy, PO Box 1364, Fairfax, VA, 22038-1364, US

(73) Assignee: K-JUMP HEALTH CO., LTD.

(21) Appl. No.: 11/898,009

(22) Filed: Sep. 7, 2007

(51) Int. Cl. A61B 5/02

(52) U.S. Cl. 600/493

(57) ABSTRACT

An externally-connected expandable blood pressure meter includes an electronic blood pressure meter which has a measuring unit to output an inspected signal based on a measured blood pressure and a processing unit to receive the inspected signal and execute signal transformation according to an output interface, and an external connection port located on the electronic blood pressure meter and electrically connected to the processing unit to provide a single expansion output device to output inspected results contained in the inspected signal. The output interface is located on the expansion output device.

### FIELD OF THE INVENTION

The present invention relates to a blood pressure meter and particularly to an externally-connected expandable blood pressure meter.

### BACKGROUND OF THE INVENTION

The conventional mercury blood pressure meter has a glass tube containing mercury to measure blood pressure. It is easily broken and damaged by impact when subject to external forces. Thus an electronic blood pressure instrument has been developed as shown in FIG. 1. It has a measuring unit 10 to inspect human blood pressure through gas, a display screen 14 to display inspected results, a processing unit 12 to perform data processing, an operation interface 18 for user operation, a printer 20 to output the inspected results through paper, a storage device 26 to record the inspected results, and a loud speaker 28 to output the inspected results through voice or sound. All the aforesaid elements are integrated into one set to provide multiple functions. As the prevailing trend of industrial design focuses on slim and light, the integrated multi-function electronic blood pressure instrument requires many elements and results in a complicated structure. Carrying and transportation are difficult. Moreover, the multi-function design and structure increase the cost and become more expensive. It is not affordable for many consumers. Hence another type of electronic blood pressure instrument has been developed as shown in FIG. 2. It has a plurality of external connection ports 171-17N to provide flexible expansion by selectively connecting individual external devices such as the printer 20, storage device 26 or loud speaker 28. Thus it can better meet user's requirement.

However, the external connection ports are designed differently in response to output interfaces of different expansion output devices. They not only occupy more space and contradict the slim and light requirement, also increase the cost. Hence how to improve the aforesaid problems occurred to the conventional electronic blood pressure instruments is a big issue in the industry.

### SUMMARY OF THE INVENTION

The primary object of the present invention is to integrate external connection ports of different output interfaces of an electronic blood pressure meter into a single external connection port.

To achieve the foregoing object the externally-connected expandable blood pressure meter according to the invention includes an electronic blood pressure meter which has a measuring unit to output an inspected signal based on a measured blood pressure and a processing unit to receive the inspected signal and execute signal transformation according to an output interface, and an external connection port located on the electronic blood pressure meter to provide a single expansion output device to output inspected results contained in the inspected signal. The external connection port is electrically connected to the processing unit. The output interface is located in the expansion output device.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 is a schematic view of the structure of a conventional electronic blood pressure meter.

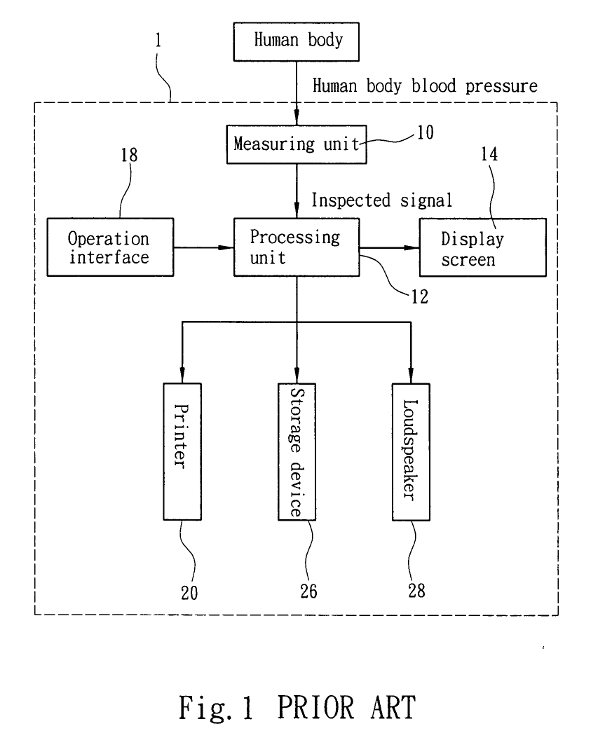

FIG. 2 is a schematic view of the structure of another conventional electronic blood pressure meter.

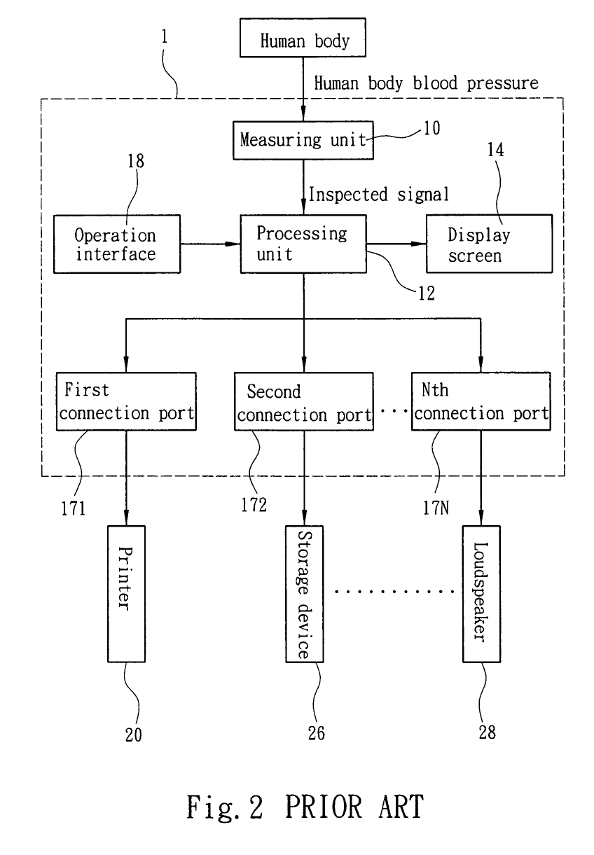

FIG. 3 is a schematic view of the structure of an embodiment of the invention.

FIG. 4 is a schematic view of the structure of another embodiment of the invention.

### DETAILED DESCRIPTION OF THE PREFERRED EMBODIMENTS

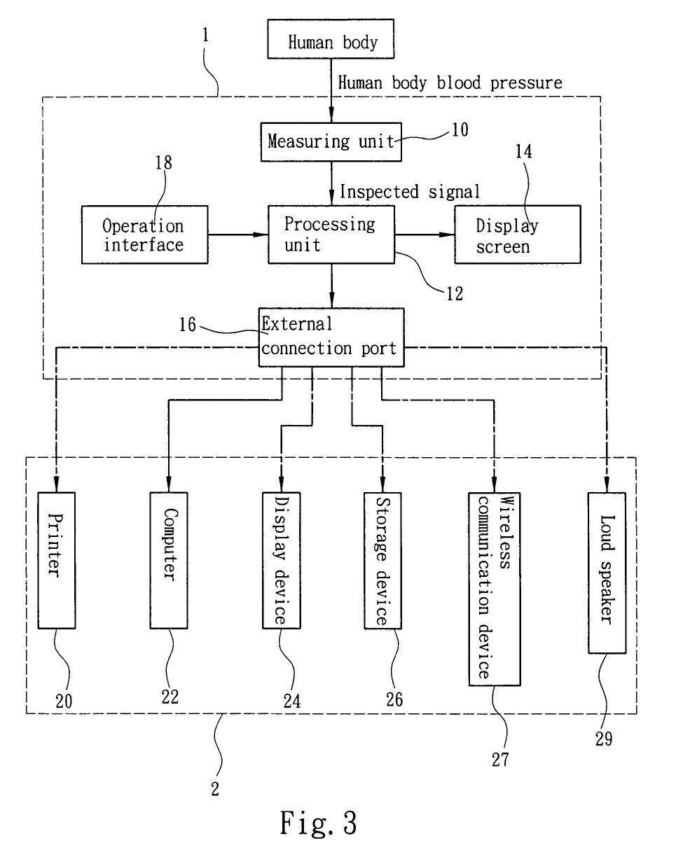

Please refer to FIG. 3 for the structure of an embodiment of the invention. The invention provides an externally-connected expandable blood pressure meter. It includes an electronic blood pressure meter 1 which has a measuring unit 10 to output an inspected signal based on a measured blood pressure and a processing unit 12 to receive the inspected signal and execute signal transformation according to an output interface (not shown in the drawings). In this embodiment the electronic blood pressure meter 1 further has a display screen 14 to directly display inspected results contained in the inspected signal. The measuring unit 10 includes an inflation unit (not shown in the drawings) which can generate compressed air by squeezing of a user or through an air pump. The inflation unit also is coupled with a check valve (not shown in the drawings) to prevent the compressed air from escaping.

The invention further includes an external connection port 16 located on the electronic blood pressure meter 1 to provide a single expansion output device 2 to output the inspected results contained in the inspected signal. The external connection port 16 may be an Universal Serial Bus (USB) or a firewire high speed serial transmission port (Firewire or IEEE 1394). The external connection port 16 is electrically connected to the processing unit 12. The output interface is located in the expansion output device 2. The expansion output device 2 may be a printer 20 to output the inspected results through paper, a computer 22 to analyze the inspect results or execute other data processing, a display device 24 to display the inspected results through images, a wireless communication device 27 to transmit the inspected results in a wireless fashion, a storage device 26 to record the inspected results, or a loud speaker 29 to output the inspected results through voice or sound.

The electronic blood pressure meter 1 also may be coupled with an operation interface 18 to control operations, such as setting power ON/OFF of the electronic blood pressure meter 1, connecting or removing the expansion output device 2, starting measurement of the measuring unit 10 for human blood pressure, or switching display pictures such as numerals or graphics on the display screen 14

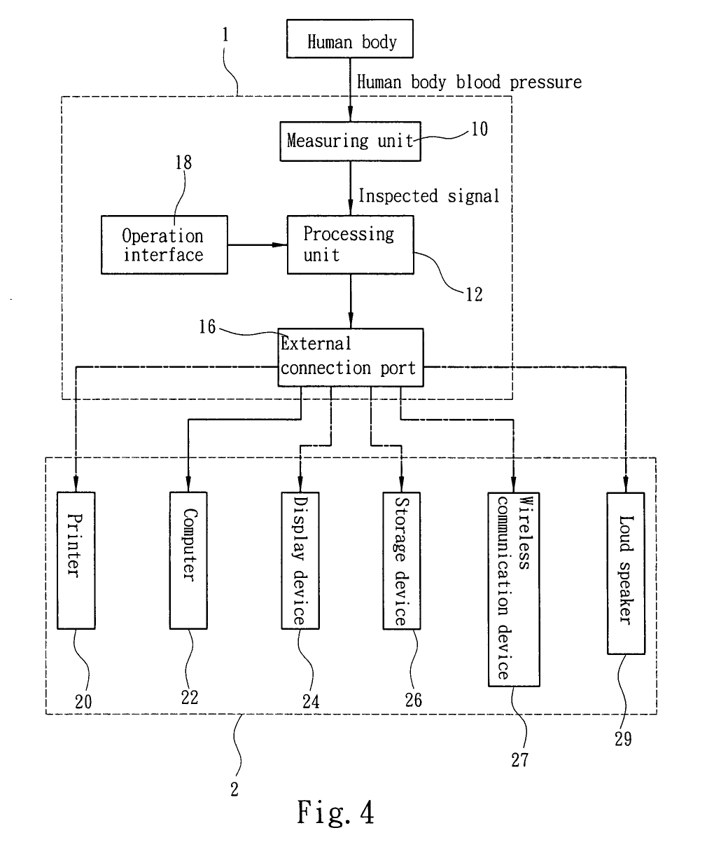

In this embodiment the electronic blood pressure meter 1 contains the display screen 14 to maintain the basic output requirement. However, in practice the display screen 14 may be dispensed with (referring to FIG. 4) to make the electronic blood pressure meter 1 more compact to meet slim and light requirement.

Thus the invention not only can reduce the space and cost associated with multiple external connection ports occurred to the conventional techniques, users can selectively carry the required expansion output device 2 without the need of carrying and using the expansion output device 2 at the destination. Transportation cost is lower. Moreover, the users can purchase the expansion output device 2 according to their requirements without incurring too much expense. By providing the external connection mode for the expansion output device 2, the electronic blood pressure meter 1 also can be designed and made slimmer and lighter. And the users can flexibly make expansion according to individual requirements.

When in use, first, install the measuring unit 10 at a desired location that is ready to measure user's blood pressure; turn on electric power through the operation interface 18 to activate the electronic blood pressure meter and the measuring unit 10 to start blood pressure measuring process; the measuring unit 10 sends an inspected signal to the processing unit 12 which informs the user through the operation interface 18 according to a selected means. For instance, if the basic display screen 14 is chosen, the processing unit 12 transforms the inspected signal to a display format to be displayed on the display screen 14 that also contains the inspected results. If the expansion output device 2 is needed to output the inspected results contained in the inspected signal, first, connect the expansion output device 2 to the external connection port 16, then select through the operation interface 18 one of the expansion output devices to output the inspected results. When the operation is finished, remove the connection with the expansion output device 2 through the operation interface 18, and separate the expansion output device 2 from the external connection port 16. Finally turn off the electronic blood pressure meter to stop operation through the operation interface 18.

As a conclusion, as the invention integrates a plurality of external connection ports for different output interfaces into a single external connection port 16, users can select and procure by themselves the required expansion output device 2. The space required is smaller and carrying is easier, and the cost also is lower due to fewer external connection ports are required. Production and transportation costs also can be reduced because of fewer expansion output devices attached to the blood pressure meter. In addition, through the externally-connected expansion output device 2, the electronic blood pressure meter 1 can be made slimmer and lighter, and users can expand flexibly to meet individual requirements. It provides a significant improvement over the conventional techniques.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

What is claimed is:

1. An externally-connected expandable blood pressure meter, comprising:
an electronic blood pressure meter which has a measuring unit to output an inspected signal based on a measured blood pressure and a processing unit to receive the inspected signal and execute signal transformation according to an output interface; and
an external connection port which is located on the electronic blood pressure meter and electrically connected to the processing unit to provide a single expansion output device to output inspected results contained in the inspected signal, the output interface being located on the expansion output device.

2. The externally-connected expandable blood pressure meter of claim 1, wherein the expansion output device is a printer.

3. The externally-connected expandable blood pressure meter of claim 1, wherein the expansion output device is a sound speaker.

4. The externally-connected expandable blood pressure meter of claim 1, wherein the expansion output device is a computer.

5. The externally-connected expandable blood pressure meter of claim 1, wherein the expansion output device is a display device.

6. The externally-connected expandable blood pressure meter of claim 1, wherein the expansion output device is a wireless communication device.

7. The externally-connected expandable blood pressure meter of claim 1, wherein the expansion output device is a display screen.

8. The externally-connected expandable blood pressure meter of claim 1, wherein the expansion output device is a Universal Serial Bus.

9. The externally-connected expandable blood pressure meter of claim 1, wherein the expansion output device is a firewire high speed serial transmission port.
